(19) United States
(12) Patent Application Publication
(10) Pub. No.: US 2023/0007883 A1
(43) Pub. Date: Jan. 12, 2023

(54) INJECTION PUMP NEEDLE MECHANICS

(71) Applicant: JANSSEN BIOTECH, INC., Horsham, PA (US)

(72) Inventors: Steven M. VESOLE, San Bruno, CA (US); Brandon MCKENZIE, West Chester, PA (US); Scott UHLAND, Sudbury, MA (US)

(21) Appl. No.: 17/785,326

(22) PCT Filed: Nov. 16, 2020
(86) PCT No.: PCT/IB2020/060751

§ 371 Date: Jun. 14, 2022

Related U.S. Application Data
(60) Provisional application No. 62/950,376, filed on Dec. 19, 2019.

(51) Int. Cl. A61M 5/142; A61M 5/158

(52) U.S. Cl. CPC A61M 5/142; A61M 5/158; A61M 5/3286

(57) ABSTRACT

Various exemplary methods, systems, and devices for injection pump needle mechanics are provided.

### FIELD

The present disclosure relates generally to injection pump needle mechanics.

### BACKGROUND

Pharmaceutical products (including large and small molecule pharmaceuticals, hereinafter “drugs”) are administered to patients in a variety of different ways for the treatment of specific medical indications. A pump is a type of drug administration device that can administer a liquid drug to the patient. Some pumps are wearable by a patient and can include a reservoir, such as a vial or a cartridge, that contains the liquid drug therein for delivery to the patient through a needle inserted into tissue of the patient. However, delivering the drug through the needle can cause various adverse effects, such as patient pain and tissue inflammation.

Accordingly, there remains a need for improved liquid drug pumps.

### SUMMARY

In general, methods, systems, and devices for injection pump needle mechanics are provided.

In one aspect, a pump configured to deliver a drug to a patient is provided that in one embodiment includes a reservoir configured to contain a liquid drug therein, a needle including a distal tip configured to be inserted into a patient and configured to reduce pressure at the distal tip of the needle, and a pumping assembly configured to drive the liquid drug from the reservoir and into the needle for delivery of the liquid drug into the patient. The pump can have any number of variations.

In another aspect, a method of using a pump configured to deliver a drug to a patient is provided and in one embodiment includes activating a pumping assembly of the pump to move a liquid drug from a reservoir of the pump and into a needle of the pump. The method can have any number of variations.

### BRIEF DESCRIPTION OF DRAWINGS

The present invention is described by way of reference to the accompanying figures which are as follows:

FIG. 1 is a schematic view of an embodiment of a pump configured to deliver a liquid drug to a patient;

FIG. 2 is a schematic view of another embodiment of a pump configured to deliver a liquid drug to a patient and an embodiment of a reservoir configured to be received in the pump;

FIG. 3 is a schematic view of the reservoir and pump of FIG. 2 coupled together;

FIG. 4 is a schematic view of the reservoir and pump of FIG. 3 with a conduit of the pump penetrated into the reservoir;

FIG. 5 is a schematic view of yet another embodiment of a pump configured to deliver a liquid drug to a patient;

FIG. 6 is a perspective view of an embodiment of a needle injecting a liquid drug into tissue;

FIG. 7 is a table showing information for seven liquid injection simulation runs;

FIG. 8 is a graphic view of a blunt tip needle of runs 1-3 of FIG. 7;

FIG. 9 is a graphic view of a spherical tip needle of runs 4 and 5 of FIG. 7;

FIG. 10 is a graphic view of a 10° beveled tip needle of run 6 of FIG. 7;

FIG. 11 is a graphic view of a 20° beveled tip needle of run 7 of FIG. 7;

FIG. 12 is a graph of pressure versus time for run 1 of FIG. 7;

FIG. 13 is a graph of pressure versus time for run 2 of FIG. 7;

FIG. 14 is a graph of pressure versus time for run 3 of FIG. 7;

FIG. 15 is a graph of pressure versus time for run 4 of FIG. 7;

FIG. 16 is a graph of pressure versus time for run 5 of FIG. 7;

FIG. 17 is a graph of pressure versus time for run 6 of FIG. 7;

FIG. 18 is a graph of pressure versus time for run 7 of FIG. 7;

FIG. 19 is a perspective view of a blunt tip needle with side exit openings of an eighth liquid injection simulation run;

FIG. 20 is a quartered view of the needle of FIG. 19 positioned in tissue;

FIG. 21 is a graph of pressure versus time for run 8 of FIG. 19;

FIG. 22 is a perspective view of a blunt tip needle with side exit openings of a ninth liquid injection simulation run;

FIG. 23 is a quartered view of the needle of FIG. 22 positioned in tissue;

FIG. 24 is a graph of pressure versus time for run 9 of FIG. 22;

FIG. 25 is a graphic view of drug distribution for run 2 of FIG. 7;

FIG. 26 is a graphic view of drug distribution for run 8 of FIG. 19;

FIG. 27 is a graphic view of drug distribution for run 9 of FIG. 22; and

FIG. 28 is a graphic view of tissue of runs 1-7 of FIG. 7, run 8 of FIG. 19, and run 9 of FIG. 22.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Various exemplary methods, systems, and devices for injection pump needle mechanics are provided.

The drug to be delivered using a pump as described herein can be any of a variety of drugs. Examples of drugs that can be delivered using a pump as described herein include an antibodies (such as monoclonal antibodies), hormones, antitoxins, substances for the control of pain, substances for the control of thrombosis, substances for the control of infection, peptides, proteins, human insulin or a human insulin analogue or derivative, polysaccharide, DNA, RNA, enzymes, oligonucleotides, antiallergics, antihistamines, anti-inflammatories, corticosteroids, disease modifying anti-rheumatic drugs, erythropoietin, and vaccines.

The needle mechanics described herein can be used with a variety of drug delivery pumps configured to deliver a drug to a patient. Examples of drug delivery pumps include the pumps described in Intl. Pat. Pub. WO 2018/096534 entitled “Apparatus For Delivering A Therapeutic Substance” published May 31, 2018, in U.S. Pat. Pub. No. 2019/0134295 entitled “Local Disinfection For Prefilled Drug Delivery System” published May 9, 2019, in U.S. Pat. No. 7,976,505 entitled “Disposable Infusion Device Negative Pressure Filling Apparatus And Method” issued Jul. 12, 2011, and in U.S. Pat. No. 7,815,609 entitled “Disposable Infusion Device Positive Pressure Filling Apparatus And Method” issued Oct. 19, 2010, which are hereby incorporated by reference in their entireties. Other examples of drug delivery pumps include the SmartDose® Drug Delivery Platform available from West Pharmaceutical Services, Inc. of Exton, Pa., the OMNIPOD® available from Insulet Corp. of Acton, Mass., the YpsoDose® patch injector available from Ypsomed AG of Burgdorf, Switzerland, the BD Libertas™ wearable injector available from Becton, Dickinson and Co. of Franklin Lakes, N.J., the Sorrel Medical pump available from Sorrel Medical of Netanya, Israel, the SteadyMed PatchPump® available from SteadyMed Ltd. of Rehovot, Israel, the Sensile Medical infusion pump available from Sensile Medical AG of Olten, Switzerland, the SonceBoz wearable injectors available from SonceBoz SA of Sonceboz-Sombeval, Switzerland, enFuse® available from Enable Injections of Cincinnati, Ohio, the on-body injector for Neulasta® available from Amgen, Inc. of Thousand Oaks, Calif., the Pushtronex® System available from Amgen, Inc. of Thousand Oaks, Calif., and the Imperium® pump available from Unilife Corp. of King of Prussia, Pa.

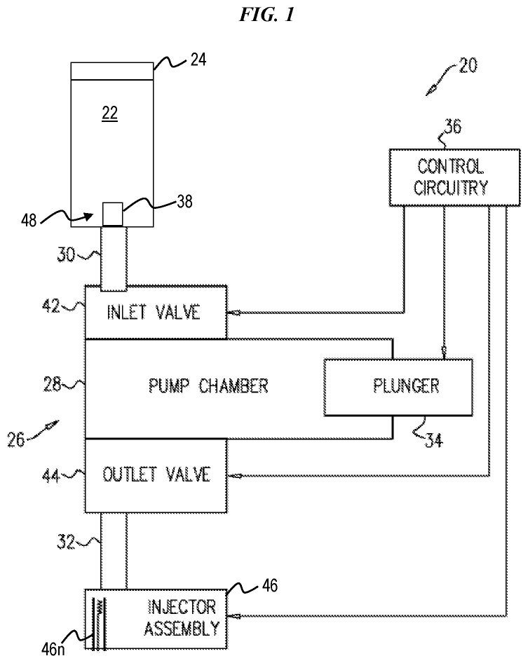

FIG. 1 illustrates an embodiment of a pump 20, e.g., a patch pump, configured to be worn by a patient and to deliver a drug (also referred to herein as a “therapeutic substance”) 22 to the patient. The pump 20 can be configured to be attached to the patient in any of a variety of ways, as will be appreciated by a person skilled in the art, such as by including a backing or label configured to be removed from a body of the pump 20 to expose adhesive attachable to the patient. The pump 20 includes a therapeutic substance reservoir 24 containing the drug 22 therein. The reservoir 24 can be prefilled by a medical vendor or device manufacturer, or the reservoir 24 can be filled by a user (e.g., the patient, the patient's caregiver, a doctor or other health care professional, a pharmacist, etc.) prior to use of the pump 20. Alternatively, the reservoir 24 can come prefilled from a medical vendor ready to be loaded or inserted into pump 20 prior to use. The pump 20 also includes a conduit 38 through which the drug 22 is configured to pass from the reservoir 24 and into an inlet fluid path 30 operatively connected to an injector assembly 46 of the pump 20 that is configured to deliver the therapeutic substance 22 into a patient. The conduit 38 is thus a tube in which the drug 22 can flow.

The electromechanical pumping assembly 26, e.g., a motor thereof, is operatively connected to the reservoir 24 and is configured to cause delivery of the therapeutic substance 22 to the patient via the injector assembly 46, e.g., through a needle 46n of the injector assembly 46 that has been inserted into tissue of the patient. The electromechanical pumping assembly 26 is shaped to define a rigid pump chamber 28 that includes a therapeutic substance inlet 30 through which the therapeutic substance 22 is received from the conduit 30, and hence from the reservoir 24, into the pump chamber 28. The rigid pump chamber 28 also includes a fluid path outlet 32 through which the therapeutic substance 22 is delivered from the pump chamber 28 to the patient via the injector assembly 46. Although the pumping assembly 26 is electromechanical in this illustrated embodiment, the pumping assembly of the pump 20 (and for other embodiments of pumps described herein) can instead be mechanical. The mechanical pumping assembly need not include any electronic components or controls. For example, the mechanical pumping assembly can include a balloon diaphragm configured to be activated to cause delivery of a drug through mechanical action.

The pump 20 also includes a plunger 34 slidably disposed within the pump chamber 28 and sealably contacting an inside of the pump chamber 28. The plunger 34 is configured to be in direct contact with the drug 22 in the pumping chamber 28.

The pump 20 also includes control circuitry 36. The electromechanical pumping assembly 26 is configured to be driven to operate in two pumping phases by the control circuitry 36. In a first pumping phase, the control circuitry 36 is configured to drive the plunger 34 (e.g., slidably move the plunger 34 in the pump chamber 28) to draw the drug 22 from the reservoir 24 into the conduit 38, then into the inlet fluid path 30, then through an inlet valve 42 and into the pump chamber 28. The inlet valve 42 is configured to be opened and closed such that when the inlet valve 42 is open there is fluid communication between the reservoir 24 and the pump chamber 28, and when the inlet valve 42 is closed there is no fluid communication between the reservoir 24 and the pump chamber 28. During the first pumping phase, the control circuitry 36 is configured to cause the inlet valve 42 to open, cause an outlet valve 44 to close, and drive the plunger 34 to draw the therapeutic substance 22 from the reservoir 24 into the pump chamber 28, e.g., the control circuitry 36 is configured to set the inlet valve 42 and the outlet valve 44 such that the therapeutic substance 22 can flow only between the reservoir 24 and the pump chamber 28. Thus, as the plunger 34 is drawn back, therapeutic substance 22 is drawn into pump chamber 28. The control circuitry 36 causing the inlet valve 42 to open and the outlet valve 44 to close can be active control or can be passive control in which the valves 42, 44 are mechanical valves that automatically open/close due to the driving of the plunger 34.

The needle 46n of the injector assembly 46 is configured to move from inside the pump's housing to at least partially outside of the pump's housing for penetration into a patient. The electromechanical pumping assembly 26, e.g., the motor thereof as controlled by the control circuitry 36, is configured to cause the movement of the needle 46n. The needle 46n movement can occur during the first pumping phase or before the first pumping phase. In other embodiments, the needle 46n begins outside of the pump's housing.

In a second pumping phase, the control circuitry 36 is configured to drive the plunger 34 to deliver the drug 22 from the pump chamber 28 through the outlet valve 44 to the outlet fluid path 32 and then to the injector assembly 46 for delivery into the patient through the needle 46n. The outlet valve 44 is configured to be opened and closed such that when the outlet valve 44 is open there is fluid communication between the pump chamber 28 and the patient, and when the outlet valve 44 is closed there is no fluid communication between the pump chamber 28 and the patient. During the second pumping phase, the control circuitry 36 is configured to cause the inlet valve 42 to close, cause the outlet valve 44 to open, and drive the plunger 34 to deliver the therapeutic substance 22 from the pump chamber 28 in a plurality of discrete motions of the plunger 34. For example, the control circuitry 36 can be configured to set the inlet valve 42 and the outlet valve 44 such that the therapeutic substance 22 can flow only between the pump chamber 28 and the patient, and the plunger 34 is incrementally pushed back into the pump chamber 28 in a plurality of discrete motions thereby delivering the therapeutic substance 22 to the patient in a plurality of discrete dosages. Similar to that discussed above, the control circuitry 36 causing the inlet valve 42 to close and the outlet valve 44 to open can be active control or can be passive control in which the valves 42, 44 are mechanical valves that automatically open/close due to the driving of the plunger 34.

In some embodiments, the control circuitry 36 is configured to drive the plunger 34 to draw the therapeutic substance 22 into the pump chamber 28 in a single motion of the plunger 34, e.g., the plunger 34 is pulled back in a single motion to draw a volume of the therapeutic substance 22 into the pump chamber 28 during the first pumping phase. Alternatively, the control circuitry 36 can be configured to drive the plunger 34 to draw the therapeutic substance 22 into the pump chamber 28 in one or more discrete expansion motions of the plunger 34, e.g., the plunger 34 can be pulled halfway out of the pump chamber 28 in one motion and then the rest of the way out of the pump chamber 28 in a second, separate motion. In this case, a duration of some or all expansion motions of the plunger 34 during the first pumping phase are typically longer than a duration of any one of the plurality of discrete motions of the plunger 34 during the second pumping phase.

In other embodiments, the control circuitry 36 is configured to drive the plunger 34 such that a duration of the first pumping phase and a duration of the second pumping phase are unequal. For example, a duration of the second pumping phase can be in a range of five to fifty times longer than the first pumping phase, e.g., at least ten times, thirty times, fifty times, etc. longer than a duration of the first pumping phase.

The pump 20 can also include a power supply (not shown) configured to provide power to components requiring power to operate, such as the control circuitry 36. In an exemplary embodiment, the power supply is a single power supply configured to provide power to each component of the pump 20 requiring power to operate, which may help reduce cost of the pump 20, help conserve space within the pump 20 for other components, and/or help reduce an overall size of the pump 20. The power supply can, however, include a plurality of power supplies, which may help provide redundancy and/or help reduce cost of the pump 20 since some components, e.g., the control circuitry 36, may be manufactured with an on-board dedicated power supply. In an exemplary embodiment, the power supply is on-board the pump 20, which may facilitate use of the pump 20 at any time in any location. In other embodiments, the power supply can include a mechanism configured to connect the pump 20 to an external power supply.

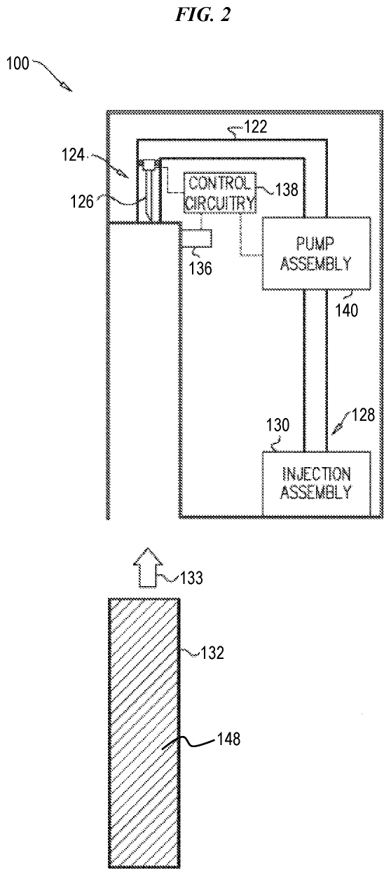

FIGS. 2-4 illustrate another embodiment of a pump 100, e.g., a patch pump, configured to be worn by a patient and to deliver a drug 148 to the patient. The pump 100 of FIGS. 2-4 is generally configured and used similar to the pump 20 of FIG. 1. The pump 100 is configured to engage with a prefilled therapeutic substance reservoir 132. Within the pump 100 is a sterile fluid path 122 for delivering a drug 148 from the reservoir 132 to a patient wearing the pump 100. The sterile fluid path 122 has a conduit 126 at an upstream end 124 of the sterile fluid path 122 and has an injection assembly (also referred to herein as an “injector assembly”) 130 at a downstream end 128 of the sterile fluid path 122.

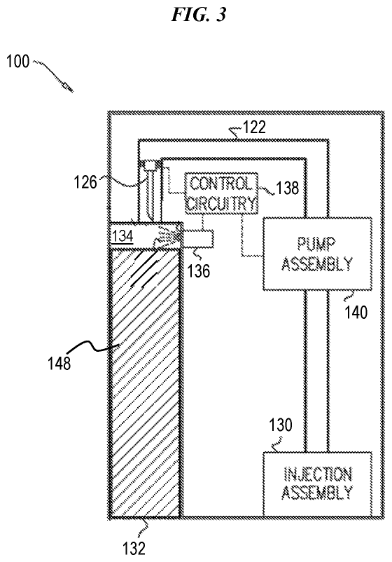

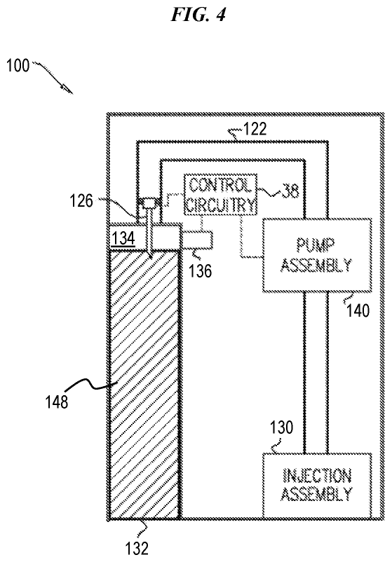

The pump 100 and the prefilled therapeutic substance reservoir 132 are configured to engage with one another, such as shown by arrow 133 in FIG. 2, e.g., the reservoir 132 is configured to be inserted into the pump 100. When the pump 100 and the reservoir 132 are engaged with one another, such as is shown in FIG. 3, a sealed disinfection chamber 134 is defined between the sterile fluid path 122 and the reservoir 132. While the pump 100 and the reservoir 132 are typically sterile, the disinfection chamber 134 is (a) initially non-sterile, and (b) typically sealed from further bacteria or virus penetration. The conduit 126 is configured to be driven to penetrate the disinfection chamber 134 and subsequently the reservoir 132 when the pump 100 and the reservoir 132 are engaged with one another, such that fluid communication is established between the reservoir 132 and the sterile fluid path 122, such as is shown in FIG. 4.

The pump 100 includes a disinfection assembly 136 configured to disinfect the disinfection chamber 134 prior to the conduit 126 penetrating the disinfection chamber 134 and thus before the conduit 126 enters the reservoir 132. The pump 100 includes control circuitry 138 configured to activate the disinfection assembly 136, to subsequently terminate the activation of the disinfection assembly 136, and to then drive the conduit 126 to penetrate the disinfection chamber 134 and subsequently the reservoir 132.

Once fluid communication is established between the reservoir 132 and the sterile fluid path 122, the control circuitry 138 is configured to drives a pump assembly 140 to draw the drug 148 from the reservoir 132 and deliver it to the patient via the injection assembly 130, e.g., via a needle thereof, similar to that discussed above regarding the control circuitry 36 and the injector assembly 46 of FIG. 1.

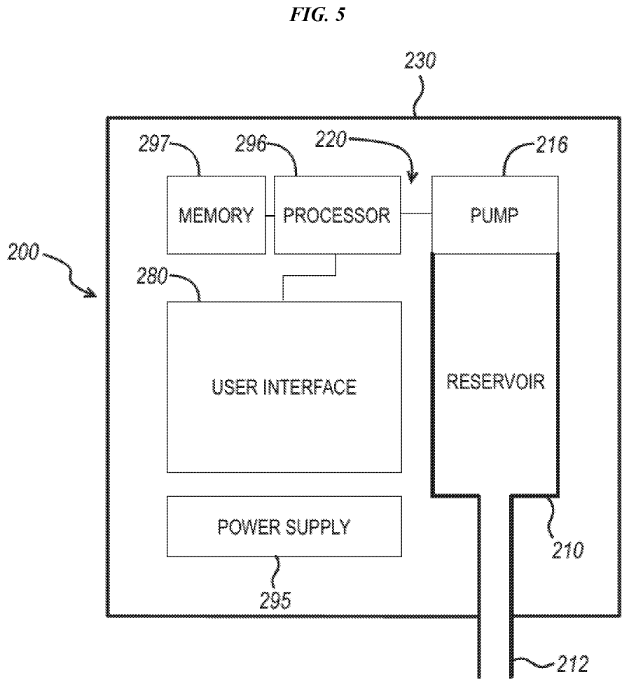

FIG. 5 illustrates another embodiment of a pump 200 configured to be worn by a patient and to deliver a drug to the patient. The pump 200 of FIG. 5 is generally configured and used similar to the pump 20 of FIG. 1. The pump 200 includes a reservoir 210 configured to contain a liquid drug therein to be delivered from the pump 200. The pump 200 also includes a pumping assembly 216 configured to cause dispensing of the drug contained in the reservoir 210 so that the drug can be delivered to the patient. The pump 200 also includes an injector assembly that includes an infusion line 212, e.g., a needle. The drug is delivered from the reservoir 210 upon actuation of the pumping assembly 216 via the infusion line 212.

The pump 200 also includes a user interface 280 configured to provide for interaction with a user. The user interface 280 can be implemented on a computer having a display screen, such as for example a cathode ray tube (CRT) or a liquid crystal display (LCD) or a light emitting diode (LED) monitor for displaying information to a user. The display screen can allow input thereto directly (e.g., as a touch screen) or indirectly (e.g., via an input device such as a keypad or voice recognition hardware and software). The user interface 280 can take the form of, e.g., a touchscreen or a keypad.

The pump 200 also includes control circuitry that includes a processor 296 and a memory 297 in operative communication with the processor 296. Actuation of the pumping assembly 216 is controlled by the processor 296, which is in operative communication with the pumping assembly 216 for controlling the pump's operation.

In at least some embodiments, the processor 296 is configured to be programmed by a user, e.g., the patient, a healthcare professional, etc., via the user interface 280. The processor 296 being user-programmable enables the pump 200 to deliver the drug to the patient in a controlled manner specific to the patient. The user can enter parameters, such as infusion duration and delivery rate, via the user interface 280, such as by the user interface 280 including a touchscreen configured to receive touch input thereto, the user interface 280 including selector button(s), and/or the user interface 280 including a keypad. The delivery rate can be set by the user to a constant infusion rate or as set intervals for periodic delivery, typically within pre-programmed limits. The programmed parameters for controlling the pumping assembly 216 are stored in and retrieved by the processor 296 from the memory 297.

The pump 200 also includes a power supply 295 configured to provide power to any components of the pump 200 that require power for operation, such as the pumping assembly 216, the processor 296, the user interface 280, and the sensor 282.

The reservoir 210, the pumping assembly 216, the user interface 280, the power supply 295, the processor 296, and the memory 297 are located within a housing (also referred to herein as a “body” of a pump) 230 of the pump 200. The infusion line 212 is partially located within the housing 230 and extends from the housing 230 for penetration into the patient. The infusion line 212 can be fixedly positioned partially within the housing 230 and partially outside the housing 230, as shown in FIG. 5, or the infusion line 212 can be movable, e.g., under control of the circuitry, from an initial position entirely within the housing 230 to a delivery position partially within the housing 230 and partially outside the housing 230.

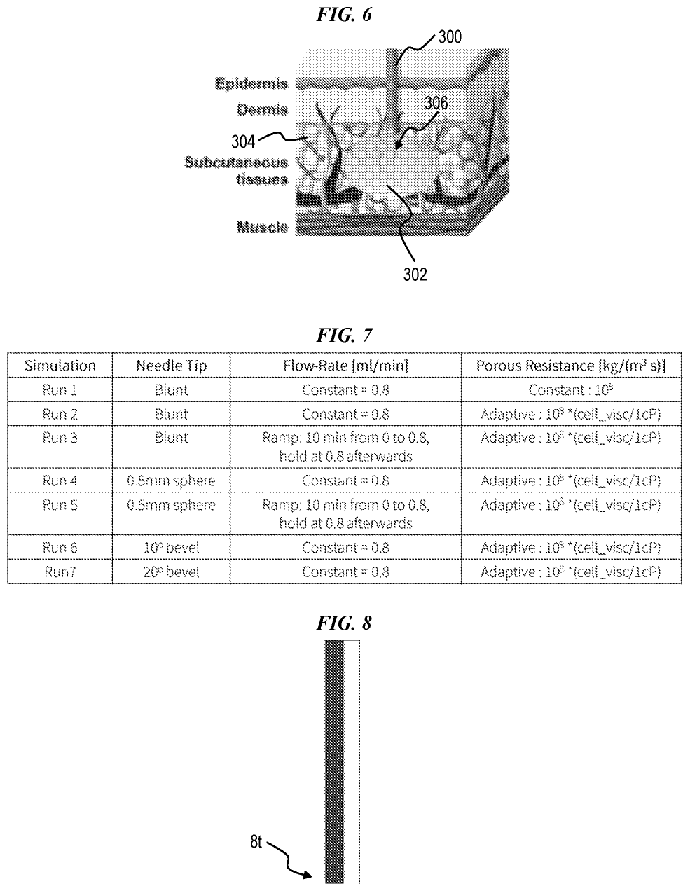

The various pumps described herein are configured to deliver a drug to a patient, e.g., the pump 20 of FIG. 1, the pump 100 of FIGS. 2-4, and the pump 200 of FIG. 5, the drug is configured to be delivered into a subcutaneous tissue or muscle of the patient through a needle of the pump's injector assembly. The drug exits the needle into the tissue or muscle through an open distal tip of the needle. For example, FIG. 6 illustrates an embodiment of a needle 300 inserted into tissue of a patient and delivering a bolus of a liquid drug 302 into subcutaneous tissue 304 through an open distal tip 306 of the needle 300.

In general, transport of a liquid drug through interstitial space will ultimately dictate drug delivery and pressure distribution in a tissue into which a needle is injecting the drug, including backpressure at the needle interface, which may contribute to patient pain. There are various factors that can affect patient comfort and successful delivery of a liquid drug into subcutaneous tissue, such as physics factors (e.g., tissue deformation such as elastic stress-strain), tissue viscoelasticity such as stress/strain versus time, drug velocity and viscosity, drug transport (e.g., Darcy's law), and needle material failure, fracture, or tearing; extrinsic tissue factors (e.g., subcutaneous and hypodermis tissue structure and thickness, and dermis tissue structure and thickness); subcutaneous tissue intrinsic properties (e.g., tissue elasticity, tissue viscoelasticity, hydraulic permeability, and interstitial fluid and solution viscosity); boundary conditions (e.g., injection speed and flow rate, absorption of drug into capillaries or lymphatic vessels, diffusion across dermis/muscle-tissue boundaries (if any), and deformation of dermal and muscle layer boundaries); drug formulation factors (e.g., molecule size and hydrophobicity); patient factors (body mass index (BMI), gender, and injection location); injection site factors (e.g., temperature and pH); and needle factors (e.g., needle geometry). With respect to tissue thickness, subcutaneous tissue thickness is highly variable, which can make it difficult to achieve patient comfort and successful delivery of a liquid drug into subcutaneous tissue.

There are various factors indicative of patient comfort and successful delivery of a liquid drug into subcutaneous tissue. For example, interstitial fluid hydrostatic pressure (buildup) can be indicative of pressure sensed on surrounding tissue and may be related to swelling, edema, bleb formation, and pain. For another example, deformation of dermal and muscle boundaries can characterize a bulge due to injection bolus. For yet another example, implementation of stress or strain based traction-separation/fracture criteria can characterize tissue damage, pain, or potential for drug leakage.

Fluid mechanics and structural models indicate that differences in tissue at an open distal tip of the needle injecting drug into the tissue, as well as the design of the needle, impact a pressure profile during injection. This pressure profile can impact inflammatory response, pain, etc. In addition, this variability can lead to wearable pump performance differences, such as higher or lower force requirements for the pump's pumping assembly that drives the drug from the pump's reservoir and out the pump's needle, the ability of the pump to operate properly, and to achieve a certain delivery profile based on power requirements and signals from the pump's control circuitry. Increased space, e.g., a void of fluid or gas, a pathway of fluid or gas, or a pocket of fluid or gas of low resistance or differing viscosity, around the distal tip of the needle can reduce injection pressures because less tissue can be displaced by the injected liquid drug.

Fluid mechanics at the needle/tissue interface thus dictates backpressure observed during injection. If the needle/tissue interface is small (e.g., the needle has a small inner diameter that defines an opening through which the drug exits the needle), the velocity gradients and resulting pressures are high. If the needle/tissue interface is made larger, tissue backpressure during injection can be significantly reduced. Placing beveled openings at the distal tip of the needle increases the flow area, reduces the velocity gradients, and reduces the pressures as compared to a blunt distal tip. The bevel can be, e.g., about 10° or about 20°. The inner diameter of the needle can be, e.g., about 0.5 mm. A person skilled in the art will appreciate that a value may not be precisely at a value but nevertheless be considered to be about that value because of any of a variety of factors, such as manufacturing tolerances and sensitivity of measurement equipment.

In some embodiments, a needle can have only one exit opening for the liquid drug at the needle's distal tip. In other embodiments, the needle can have an exit opening for the liquid drug at the needle's tip and at least one side exit opening for the liquid drug formed in a sidewall of the needle. The at least one side exit opening can be a slot, a hole, a slit, etc. formed in the needle's sidewall so as to allow a partial portion of liquid drug in the needle's inner lumen to exit through the at least one side exit opening and a remainder of the liquid drug in the needle's inner lumen to exit through the needle's tip opening. Each side exit opening can be a discrete opening formed through the sidewall, or a distal portion of the needle can be formed from a porous structure where the porous structure's pores define side exit openings. The at least one side exit opening can vary in size, location, and number. In still other embodiments, the needle can have at least one side exit opening for the liquid drug formed in a sidewall of the needle and not have an exit opening for the liquid drug at the needle's tip.

FIG. 7 shows a chart of seven liquid injection simulation runs. In each of the seven runs, the needle gauge was 27G RW, the total liquid drug delivery volume was 15 ml, the liquid drug viscosity was 12 cP, and the subcutaneous liquid drug viscosity was 1 cP. The needles in runs 1-7 have an inner diameter of 0.21082 mm, have an outer diameter of 0.4218 mm, have a total length of 9 mm, and sit in at 7 mm depth within the tissue. The flow rate (in ml/min) shown in FIG. 7 represents the liquid drug flow rate. The flow rate is constant in runs 1, 2, 4, 6, and 7 and is not constant in runs 3 and 5. In runs 3 and 5, the flow rate ramps upward and is then constant. The porous resistance (in kg/(m³ s)) shown in FIG. 7 represents resistance at the needle/tissue interface at which the liquid is released into tissue. The porous resistance is constant in run 1 and is not constant, e.g., is adaptive, in runs 2-7. The porous resistance not being constant indicates that viscosity of the liquid drug contributes to the porous resistance.

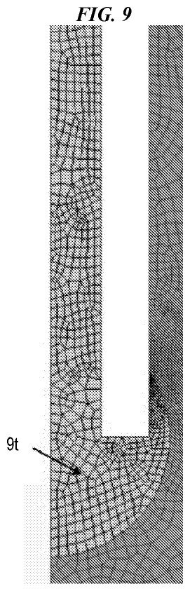

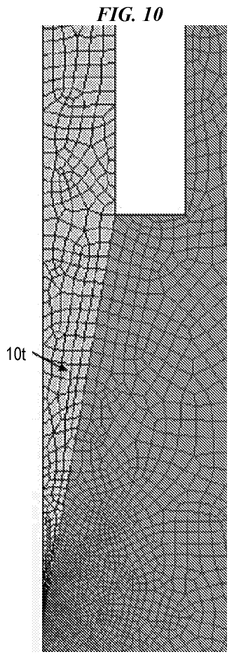

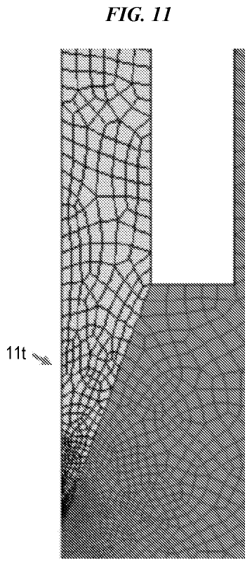

FIG. 8 illustrates the needle's blunt tip 8t of runs 1, 2, and 3. FIG. 9 illustrates the needle's spherical tip 9t of runs 4 and 5. In general, the spherical tip 9t allows for increased space around the tip 9t at the needle/tissue interface because the spherical shape moves more tissue as compared to blunt distal tips such as the blunt tip 8t. As mentioned above, increased space around the distal tip of the needle can reduce injection pressures. FIG. 10 illustrates the needle's 10° beveled tip 10t of run 6. FIG. 11 illustrates the needle's 20° beveled tip 11t of run 7.

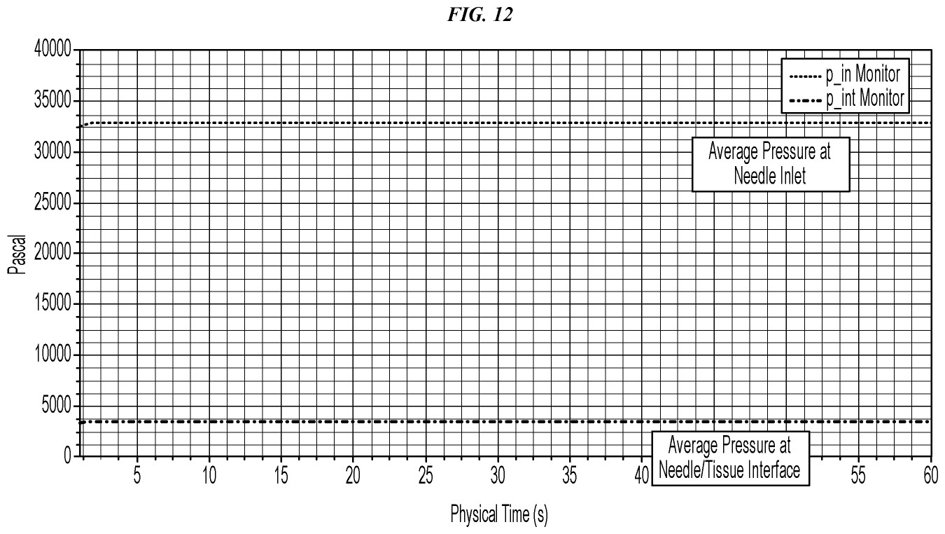

FIGS. 12-18 illustrate graphs of the pressure profiles for runs 1-7, respectively. The top line in each of the graphs represents average pressure at the needle's inlet and is thus before the liquid drug exits the needle. The bottom line in each of the graphs represents average pressure at the needle/tissue interface at which the liquid drug is released from the needle into tissue. In general, the average pressure at the needle/tissue interface being substantially constant indicates that the average pressure at the needle/tissue interface will be substantially the same for different patients and for different tissues (e.g., for drug injected into dermis tissue versus into subcutaneous tissue).

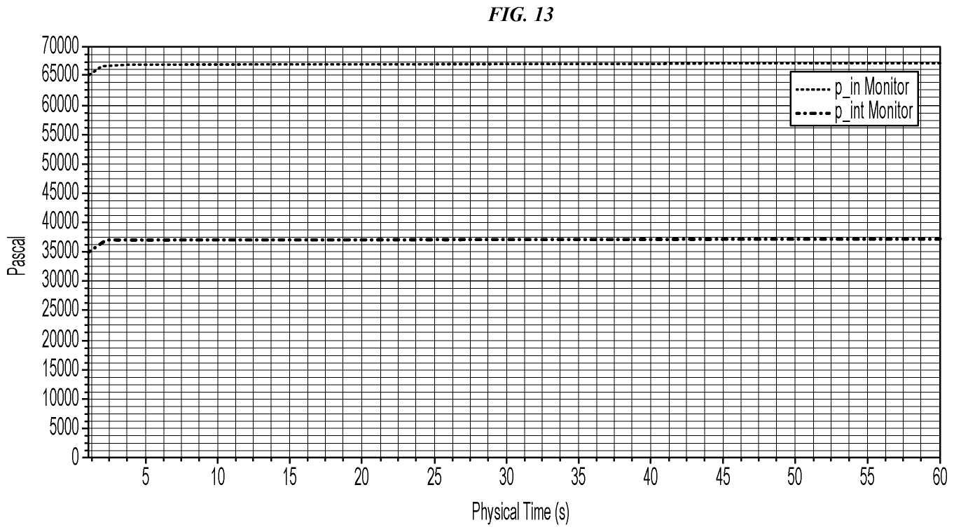

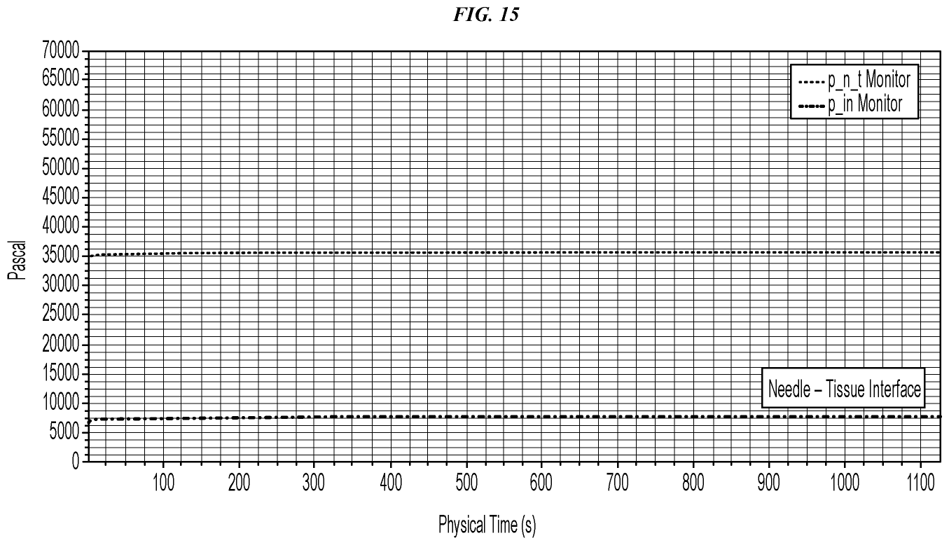

Comparing the graphs of runs 2 and 4 (FIGS. 13 and 15) demonstrates pressure profile differences for the blunt tip 8t of run 2 and the spherical tip 9t of run 4 since each of runs 2 and 4 have the same constant flow rate and the same adaptive porous resistance. FIGS. 13 and 15 show that each of the average pressure at the needle's inlet and the average pressure at the needle/tissue interface are substantially constant with the spherical tip 9t and the blunt tip 8t and are lower with the spherical tip 9t than with the blunt tip 8t.

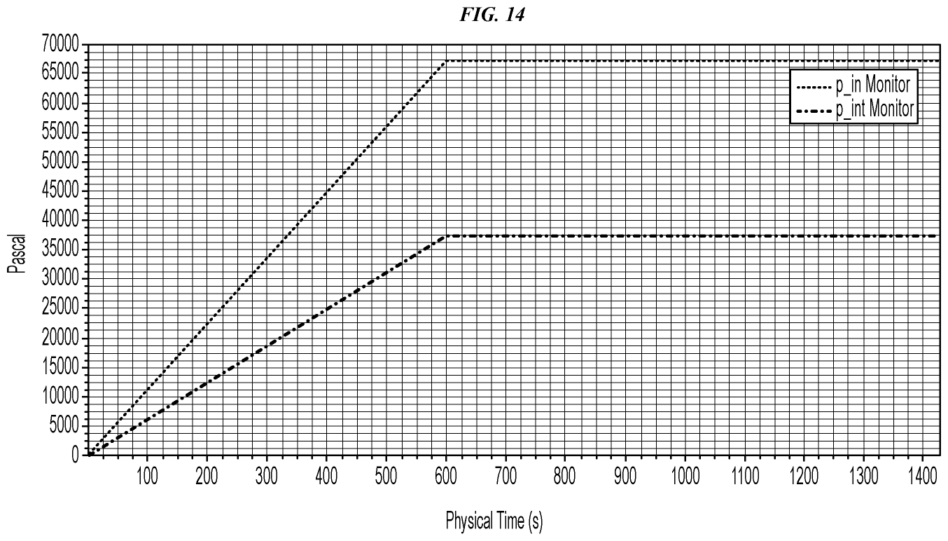

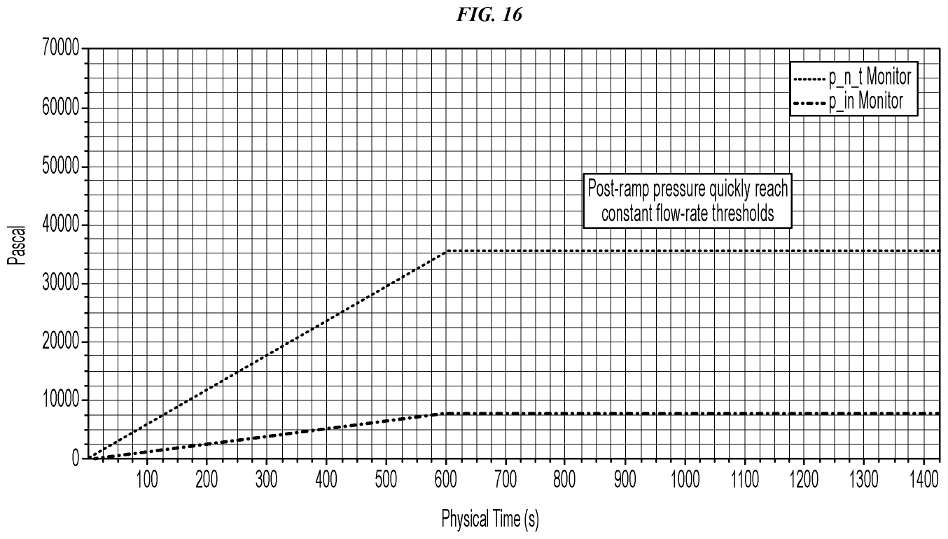

Comparing the graphs of runs 3 and 5 (FIGS. 14 and 16) demonstrates pressure profile differences for the blunt tip 8t of run 3 and the spherical tip 9t of run 5 since each of runs 3 and 5 have the same ramped and then constant flow rate and the same adaptive porous resistance. FIGS. 14 and 16 show that each of the average pressure at the needle's inlet and the average pressure at the needle/tissue interface are less steeply sloped with the spherical tip 9t than with the blunt tip 8t, are substantially constant after the ramping with the spherical tip 9t and the blunt tip 8t, and reach a lower maximum pressure with the spherical tip 9t than with the blunt tip 8t.

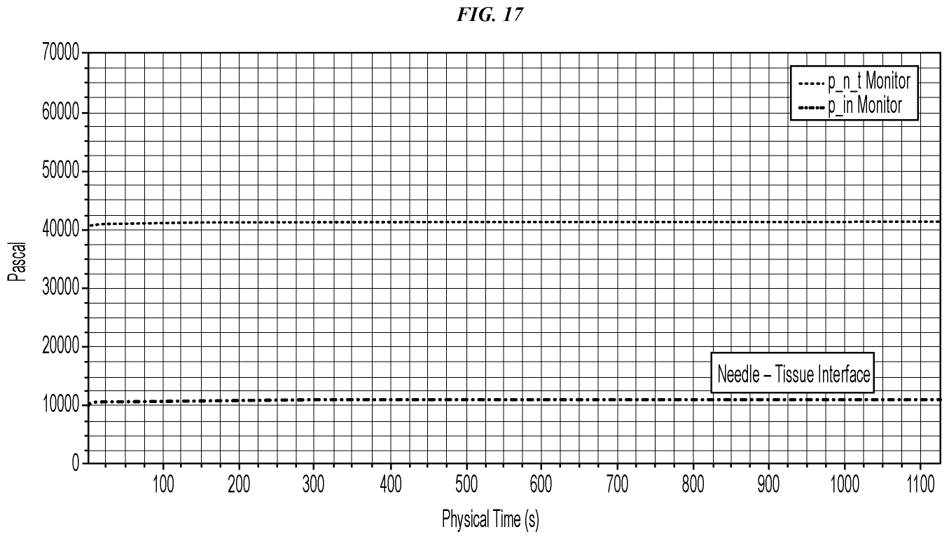

Comparing the graphs of runs 2 and 6 (FIGS. 13 and 17) demonstrates pressure profile differences for the blunt tip 8t of run 2 and the 10° beveled tip 10t of run 6 since each of runs 2 and 6 have the same constant flow rate and the same adaptive porous resistance. FIGS. 13 and 17 show that each of the average pressure at the needle's inlet and the average pressure at the needle/tissue interface are substantially constant with the 10° beveled tip 10t and the blunt tip 8t and are lower with the 10° beveled tip 10t than with the blunt tip 8t.

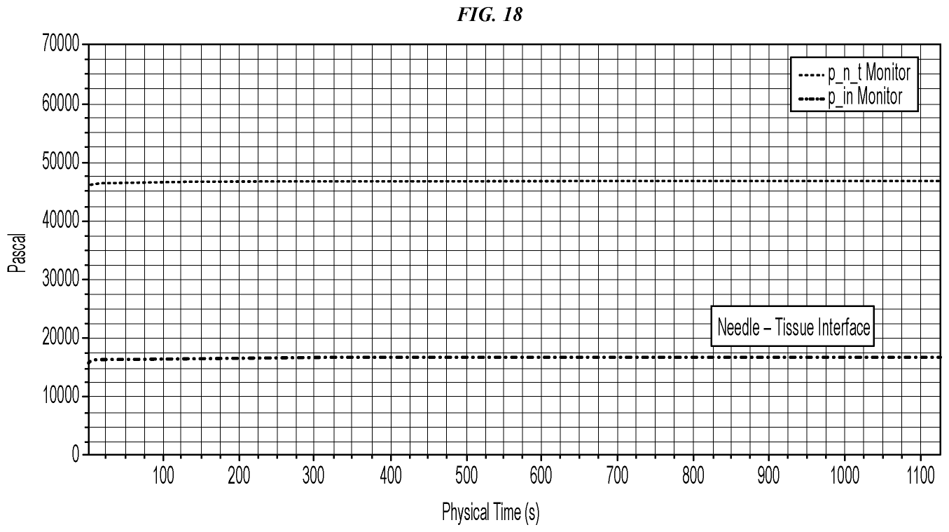

Comparing the graphs of runs 2 and 7 (FIGS. 13 and 18) demonstrates pressure profile differences for the blunt tip 8t of run 2 and the 20° beveled tip 11t of run 7 since each of runs 2 and 7 have the same constant flow rate and the same adaptive porous resistance. FIGS. 13 and 18 show that each of the average pressure at the needle's inlet and the average pressure at the needle/tissue interface are substantially constant with the 20° beveled tip 11t and the blunt tip 8t and are lower with the 20° beveled tip 11t than with the blunt tip 8t.

Comparing the graphs of runs 6 and 7 (FIGS. 17 and 18) demonstrates pressure profile differences for the different beveled tips 10t, 20t since each of runs 6 and 7 have the same constant flow rate and the same adaptive porous resistance. FIGS. 17 and 18 show that each of the average pressure at the needle's inlet and the average pressure at the needle/tissue interface are substantially constant with the 10° beveled tip 10t and the 20° beveled tip 11t and are lower with the 10° beveled tip 10t than with the 20° beveled tip 11t.

Two alternate liquid injection simulation runs, run 8 and run 9, were run for run 2 (blunt needle tip, flow rate constant at 0.8 ml/min, and adaptive porous resistance of 10⁸*(cell_vse/1 cP) kg/m³ s). The needles in runs 8 and 9 have an inner diameter of 0.210 mm, have an outer diameter of 0.42 mm, have a total length of 9 mm, and sit in at 7 mm depth within the tissue. In general, the addition of drug ports to the sides of the needle was observed to reduce injection pressures and to cause drug distributions to shift towards the skin, e.g., towards an upper portion of the subcutaneous tissue.

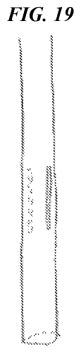

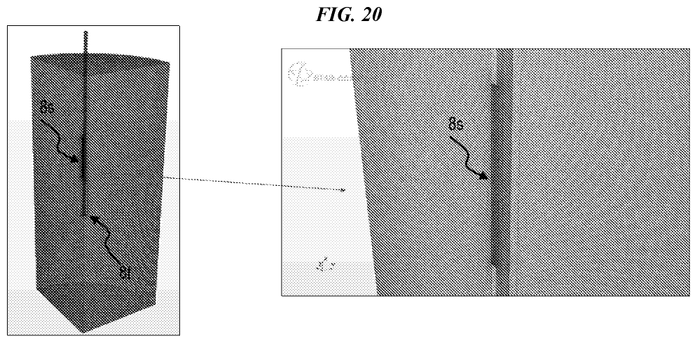

In run 8, as shown in FIGS. 19 and 20, the needle has the blunt tip 8t of run 2 and also has a plurality of side exit openings 8s formed in a sidewall of the needle. The side exit openings 8s in this illustrated embodiment are longitudinal slots. The side exit openings 8s can have a variety of locations and sizes but in this illustrated embodiment are equidistantly spaced around a circumference of the needle, have one terminal end 2 mm from the blunt tip end of the needle and extend to 4 mm from the blunt tip end of the needle, have a width of 0.05 mm, and have a length of 2 mm. Although the needle includes two side longitudinal slots 8s in this illustrated embodiment, the needle can have another number of side longitudinal slots. Additionally, the side holes can be located at more than one axial position along the needle.

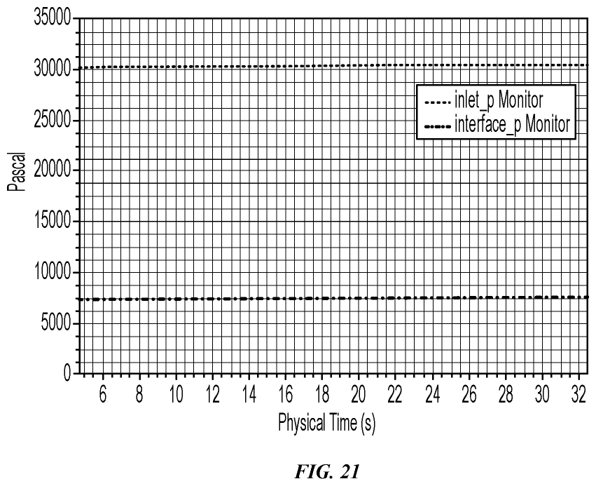

FIG. 21 illustrates a graph of the pressure profile for run 8. The top line in FIG. 21 represents average pressure at the needle's inlet and is thus before the liquid drug exits the needle. The bottom line in FIG. 21 represents average pressure at the needle/tissue interface at which the liquid drug is released from the needle into tissue.

Comparing the graphs of runs 2 and 8 (FIGS. 13 and 21) demonstrates pressure profile differences for the blunt tip 8t of run 2 with the needle having no side exit openings and the blunt tip 8t of run 9 with the needle having side exit openings in the form of slots since runs 2 and 8 are the same except for the absence (run 2) or presence (run 8) of side exit openings in the form of slots. FIGS. 13 and 21 show that each of the average pressure at the needle's inlet and the average pressure at the needle/tissue interface are substantially constant with and without the side exit openings in the form of slots and are lower with the side exit openings in the form of slots (FIG. 21) than without the side exit openings in the form of slots (FIG. 13).

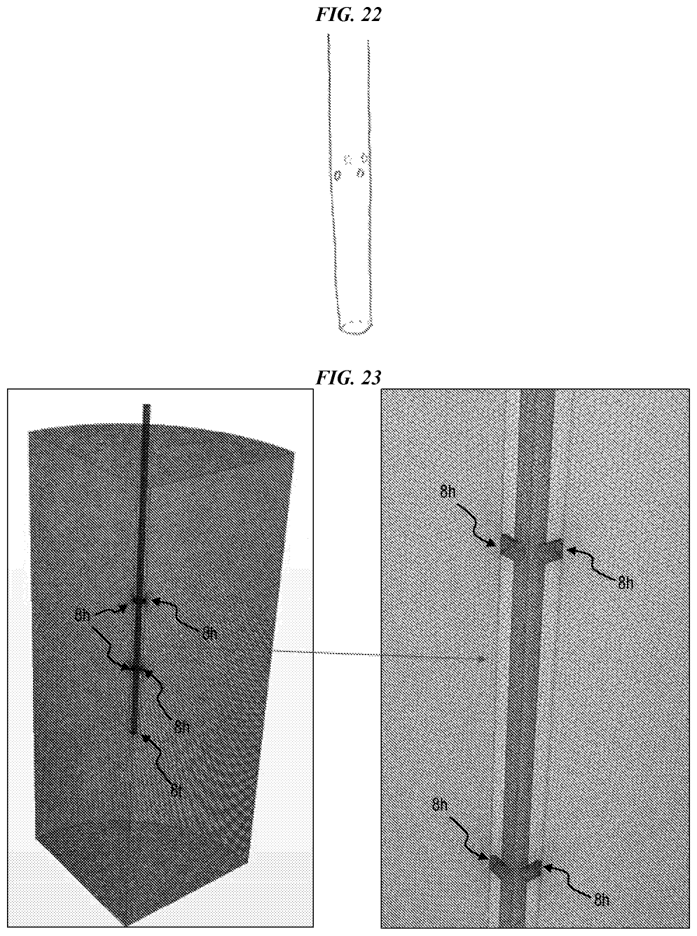

In run 9, as shown in FIGS. 22 and 23, the needle has the blunt tip 8t of run 2 and also has a plurality of side exit openings 8h formed in a sidewall of the needle. The side exit openings 8h in this illustrated embodiment are circular holes. The side exit openings 8h can have a variety of locations but in this illustrated embodiment include half the side holes 8h equidistantly spaced around a circumference of the needle at a first axial position along the needle's longitudinal axis, and half the side holes 8h equidistantly spaced around the circumference of the needle at a second, different axial position along the needle's longitudinal axis. The first and second axial positions can vary but in this illustrated embodiment are 2 mm from the blunt tip end of the needle and 4 mm from the blunt tip end of the needle. The side exit openings 8h can have a variety of sizes but in this illustrated embodiment have a diameter of 0.1 mm. Although the needle includes eight side holes 8h in this illustrated embodiment, the needle can have another number of side holes. Additionally, the side holes can be located at only one axial position along the needle or at more than two axial positions along the needle.

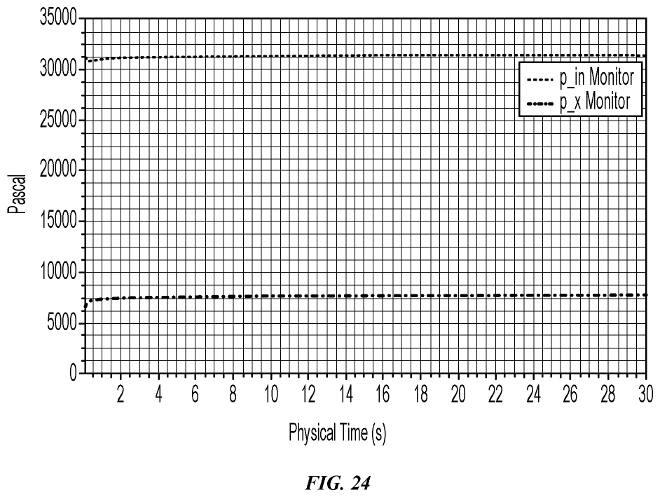

FIG. 24 illustrates a graph of the pressure profile for run 9. The top line in FIG. 24 represents average pressure at the needle's inlet and is thus before the liquid drug exits the needle. The bottom line in FIG. 24 represents average pressure at the needle/tissue interface at which the liquid drug is released from the needle into tissue.

Comparing the graphs of runs 2 and 9 (FIGS. 13 and 24) demonstrates pressure profile differences for the blunt tip 8t of run 2 with the needle having no side exit openings and the blunt tip 8t of run 9 with the needle having side exit openings in the form of holes since runs 2 and 9 are the same except for the absence (run 2) or presence (run 9) of side exit openings in the form of holes. FIGS. 13 and 24 show that each of the average pressure at the needle's inlet and the average pressure at the needle/tissue interface are substantially constant with and without the side exit openings in the form of holes and are lower with the side exit openings in the form of holes (FIG. 24) than without the side exit openings in the form of holes (FIG. 13).

Comparing the graphs of runs 8 and 9 (FIGS. 21 and 24) demonstrates pressure profile differences for the blunt tip 8t of run 8 with the needle having side exit openings in the form of slots and the blunt tip 8t of run 9 with the needle having side exit openings in the form of holes since runs 8 and 9 are otherwise the same. The average pressure at the needle/tissue interface is substantially the same in FIGS. 21 and 24, and the average pressure at the needle's inlet is lower with the side exit openings in the form of slots at a single axial position (FIG. 21) than with the side exit openings in the form of holes at two different axial positions (FIG. 24).

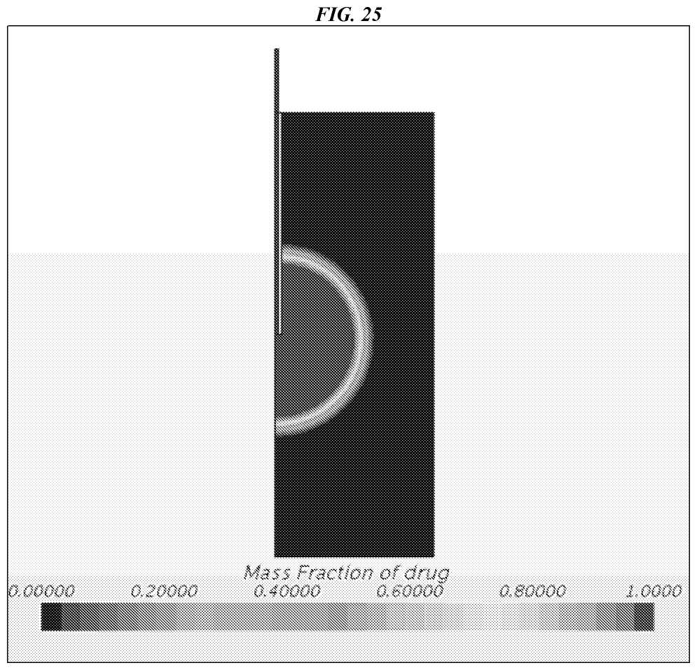

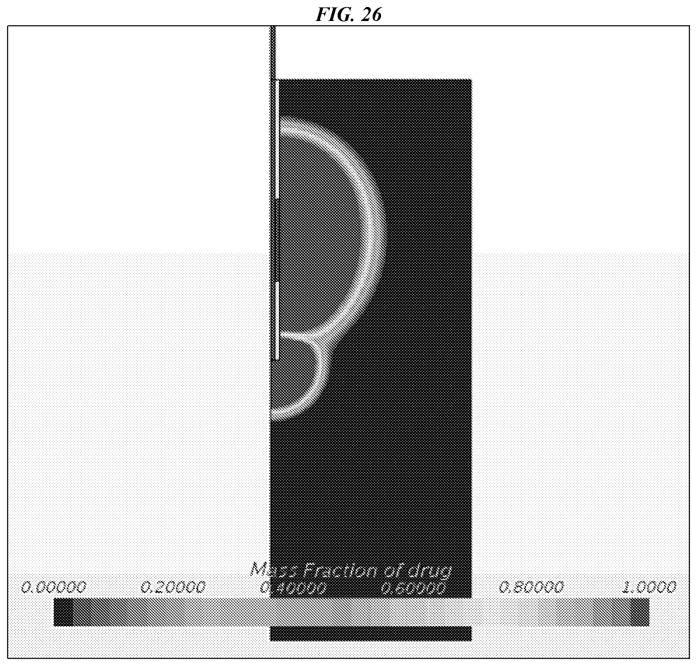

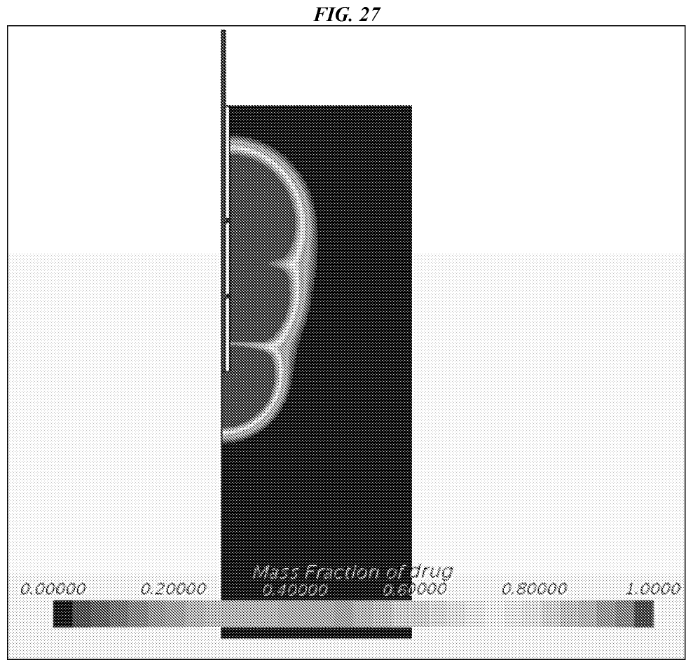

Runs 8 and 9, as compared to run 2, showed that drug distribution shifted towards the skin, e.g., towards an upper portion of the subcutaneous tissue. FIG. 25 illustrates drug distribution for run 2 at time 3 seconds. In run 2, 100% of the drug exited the needle through the blunt distal tip 8t. FIG. 26 illustrates drug distribution for run 8 at time 3 seconds. In run 8, 90% of the drug exited the needle through the side slots 8s, and 10% of the drug exited the needle through the blunt distal tip 8t. FIG. 27 illustrates drug distribution for run 9 at time 3 seconds. In run 9, 48% of the drug exited the needle through the four upper side holes 8h, 33% of the drug exited the needle through the four lower side holes 8h, and 19% of the drug exited the needle through the blunt distal tip 8t.

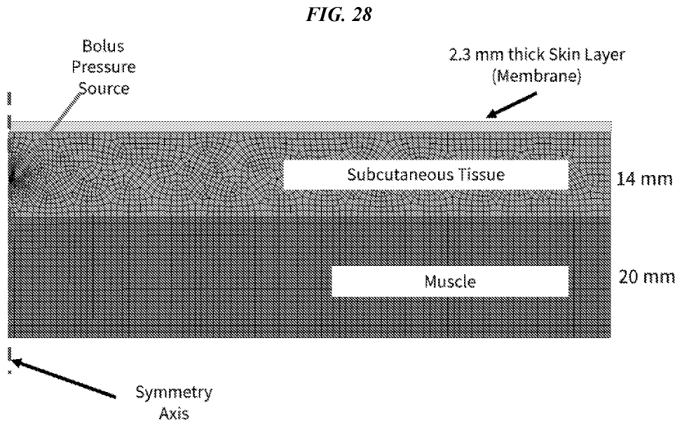

FIG. 28 illustrates the tissue for runs 1-9. The Symmetry Axis shown in FIG. 28 represents a longitudinal axis of the needle. The Bolus Pressure Source shown in FIG. 28 represents the needle/tissue interface at which the liquid drug is released from the needle into tissue, in particular into subcutaneous tissue. Lines in the subcutaneous tissue of FIG. 28 shows deformation of the subcutaneous tissue caused by the injected liquid, e.g., by the pressure applied to the tissue by the liquid. The deformation is greater the nearer the needle/tissue interface, as indicated by the more compressed lines closer to the needle/tissue interface. Muscle underlying the subcutaneous tissue is also illustrated in FIG. 28. Lines in the muscle of FIG. 28 shows that the muscle is not deforming.

A needle of the various pumps described herein, e.g., the pump 20 of FIG. 1, the pump 100 of FIGS. 2-4, and the pump 200 of FIG. 5, can be configured to reduce pressure at a distal tip of the needle. In an embodiment of a needle configured to reduce pressure at a distal tip of the needle, the needle starts at its most distal position and is configured to move passively based on resistance to flow/backpressure from tissue (in which the needle is inserted) in a proximal direction. The needle has a hub that is hydraulically coupled to a housing of the pump. The hydraulic control can be one which has a (a) positive spring that holds the needle down so that the spring is compressed when the tissue is above a certain threshold, (b) compressible spring that compresses the farther in the proximal direction the needle wants to move, the higher the tissue pressure required, (c) grease filled chamber with constant hydraulic resistance, (d) constant force spring, (e) lever arm, or (f) two magnets that oppose each other.

In another embodiment of a needle configured to reduce pressure at a distal tip of the needle, the needle starts at some position and is configured to move in laterally (left and right). Moving laterally can increase space at the needle/tissue interface, which as mentioned above can reduce injection pressures. After being moved, the needle can return to its initial position or can be in a new position. The elasticity of the tissue in which the needle is located and/or the amount of space created by the needle's movement can dictate whether the needle is in the new position or returns to its initial position. The needle has a hub that is hydraulically coupled to a housing of the pump. The hub is configured to provide for the lateral movement of the needle with a fulcrum or an intentional play of the needle in the hub. The hydraulic control can be one which has a (a) positive spring that holds the needle down so that the spring is compressed when the tissue is above a certain threshold, (b) torque spring in the hub that results in a bending moment resulting in lateral deflection, (c) compressible spring that compresses the farther in the proximal direction the needle wants to move, the higher the tissue pressure required, (d) grease filled chamber with constant hydraulic resistance, (e) constant force spring, (f) lever arm, or (g) two magnets that oppose each other.

In another embodiment of a needle configured to reduce pressure at a distal tip of the needle, the needle is configured to move actively in a distal direction or a proximal direction. Moving distally or proximally can increase space at the needle/tissue interface, which as mentioned above can reduce injection pressures. After being moved, the needle can return to its initial position or can be in a new position. The elasticity of the tissue in which the needle is located and/or the amount of space created by the needle's movement can dictate whether the needle is in the new position or returns to its initial position. The pump includes a sensor configured to monitor flow resistance. The pump's control circuitry is configured to receive data from the sensor indicative of the flow resistance. The control circuitry is configured to control the pump's pumping assembly, e.g., a motor thereof, such that the motor's torque maintains the needle at the needle's current angular position. The pump includes an encoder configured to confirm the needle's angular position and/or distal position. The pump includes electromechanical means configured to be driven by the motor (as controlled by the control circuitry) to cause selective movement of the needle proximally and distally to change a location of the needle's distal tip in the tissue and thus change a terminal end of the liquid drug flow path from the needle. The needle is electromechanically, operatively coupled to the motor via gearing, which can be independent or can be secondarily driven by a power source based on signal from the pump's control circuitry.

In another embodiment of a needle configured to reduce pressure at a distal tip of the needle, the needle is configured to move in a distal direction or a proximal direction using an active means of the pump in the form of a vibration mechanism of the pump. Moving distally or proximally can increase space at the needle/tissue interface, which as mentioned above can reduce injection pressures. The vibration mechanism is configured to allow selective distal and proximal movement of the needle against a hydraulic pressure. After being selectively moved, the needle can return to its initial position or can be in a new position. The elasticity of the tissue in which the needle is located and/or the amount of space created by the needle's movement can dictate whether the needle is in the new position or returns to its initial position. The needle has a hub that is hydraulically coupled to a housing of the pump. The hydraulic control can be one which has a (a) positive spring that holds the needle down so that the spring is compressed when the tissue is above a certain threshold, (b) compressible spring that compresses the farther in the proximal direction the needle wants to move, the higher the tissue pressure required, (c) grease filled chamber with constant hydraulic resistance, (d) constant force spring, (e) lever arm, or (f) two magnets that oppose each other. The vibration of the needle acts against the hydraulic control. Alternatively, no hydraulic control may be present, and the needle can remain fixed by vibrating against a rigid base.

In another embodiment of a needle configured to reduce pressure at a distal tip of the needle, the needle is configured to move laterally (left and right) using an active means of the pump in the form of a vibration mechanism of the pump. Moving laterally can increase space at the needle/tissue interface, which as mentioned above can reduce injection pressures. The vibration mechanism is configured to allow selective lateral movement of the needle against a hydraulic pressure. After being selectively moved, the needle can return to its initial position or can be in a new position. The elasticity of the tissue in which the needle is located and/or the amount of space created by the needle's movement can dictate whether the needle is in the new position or returns to its initial position. The needle has a hub that is hydraulically coupled to a housing of the pump. The hydraulic control can be one which has a (a) positive spring that holds the needle down so that the spring is compressed when the tissue is above a certain threshold, (b) compressible spring that compresses the farther in the proximal direction the needle wants to move, the higher the tissue pressure required, (c) grease filled chamber with constant hydraulic resistance, (d) constant force spring, (e) lever arm, or (f) two magnets that oppose each other. The vibration of the needle acts against the hydraulic control. Alternatively, no hydraulic control may be present, and the needle can remain fixed by vibrating against a rigid base.

In another embodiment of a needle configured to reduce pressure at a distal tip of the needle, a closed loop system is provided including an active means that is operatively connected to the fluid path resistance and is configured to automatically vibrate. The vibration can cause an increase in space at the needle/tissue interface, which as mentioned above can reduce injection pressures. The pump includes a sensor configured to detect clogging of the needle, such as by detecting a pressure that is above a predetermined maximum amount of acceptable pressure so as to indicate a probable clog in the needle because of a higher pressure existing than normal and/or by detecting a motor current that is above a predetermined maximum amount of acceptable motor current so as to indicate a probable clog via the motor working harder than normal. The pump's control circuitry is configured to receive data from the sensor indicative of the detected clogging. The control circuitry is configured to control the active means to vibrate in response the sensor detecting clogging of the needle. The control circuitry is configured to control the pump's pumping assembly, e.g., a motor thereof, to control the vibration of active means.

In any of the above embodiments of a needle configured to reduce pressure at a distal tip of the needle, the needle's positional changes can be during infusion or before the start of infusion, where there is a desire to create a pocket of space or area of least resistance around a terminal end of the fluid path, e.g., at a distal tip of the needle out of which the drug flows.

As discussed herein, one or more aspects or features of the subject matter described herein, for example components of the control circuitry and the user interface, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

The present disclosure has been described above by way of example only within the context of the overall disclosure provided herein. It will be appreciated that modifications within the spirit and scope of the claims may be made without departing from the overall scope of the present disclosure.

What is claimed is:

1. A pump configured to deliver a drug to a patient, comprising:
a reservoir configured to contain a liquid drug therein;
a needle including a distal tip configured to be inserted into a patient and configured to reduce pressure at the distal tip of the needle; and
a pumping assembly configured to drive the liquid drug from the reservoir and into the needle for delivery of the liquid drug into the patient.

2. The pump of claim 1, wherein the distal tip of the needle is beveled.

3. The pump of any of claim 1, wherein the needle has a hub that is hydraulically coupled to a housing of the pump using a hydraulic control.

4. The pump of claim 3, wherein the needle is configured to move passively based on resistance to flow/backpressure from tissue, in which the needle is inserted, in a proximal direction; and
the hydraulic control has one of a (a) positive spring that holds the needle down so that the spring is compressed when the tissue is above a certain threshold, (b) compressible spring, (c) grease filled chamber with constant hydraulic resistance, (d) constant force spring, (e) lever arm, or (f) two magnets that oppose each other.

5. The pump of claim 3, wherein the needle is configured to move laterally; and
the hydraulic control has one of a (a) positive spring that holds the needle down so that the spring is compressed when the tissue is above a certain threshold, (b) torque spring in the hub that results in a bending moment resulting in lateral deflection, (c) compressible spring, (d) grease filled chamber with constant hydraulic resistance, (e) constant force spring, (f) lever arm, or (g) two magnets that oppose each other.

6. The pump of claim 3, wherein the needle is configured to move actively in a distal direction or a proximal direction; and
the pump further comprises control circuitry configured to control a motor of the pumping assembly such that motor torque maintains a current angular position of the needle.

7. The pump of claim 3, wherein the pump includes a vibration mechanism configured to vibrate;
the needle is configured to move in a distal direction or a proximal direction using the vibration mechanism; and
the hydraulic control has one of a (a) positive spring that holds the needle down so that the spring is compressed when the tissue is above a certain threshold, (b) compressible spring, (c) grease filled chamber with constant hydraulic resistance, (d) constant force spring, (e) lever arm, or (f) two magnets that oppose each other.

8. The pump of claim 3, wherein the pump includes a vibration mechanism configured to vibrate;
the needle is configured to move in laterally using the vibration mechanism; and
the hydraulic control has one of a (a) positive spring that holds the needle down so that the spring is compressed when the tissue is above a certain threshold, (b) compressible spring, (c) grease filled chamber with constant hydraulic resistance, (d) constant force spring, (e) lever arm, or (f) two magnets that oppose each other.

9. The pump of claim 3, further comprising a sensor configured to detect clogging of the needle;
wherein the control circuitry is configured to receive data from the sensor indicative of the detected clogging; and
wherein the control circuitry is configured to cause active means of the pump to vibrate in response the sensor detecting clogging of the needle.

10. The pump of claim 1, further comprising control circuitry configured to cause activation of the pumping assembly and thereby move the liquid drug from the reservoir and into the needle.

11. The pump of claim 1, wherein the pump is configured to be worn by a patient.

12. The pump of claim 1, wherein the liquid drug is one of an antibody, a hormone, an antitoxin, a substance for control of pain, a substance for control of thrombosis, a substance for control of infection, a peptide, a protein, human insulin or a human insulin analogue or derivative, polysaccharide, DNA, RNA, an enzyme, an oligonucleotide, an antiallergic, an antihistamine, an anti-inflammatory, a corticosteroid, a disease modifying antirheumatic drug, erythropoietin, and a vaccine.

13. A method of using the pump of claim 1, comprising:
activating the pumping assembly to move the liquid drug from the reservoir and into the needle.

14. The method of claim 13, wherein the pump further comprises control circuitry configured to cause the activation of the pumping assembly.

15. The method of claim 13, wherein the liquid drug is one of an antibody, a hormone, an antitoxin, a substance for control of pain, a substance for control of thrombosis, a substance for control of infection, a peptide, a protein, human insulin or a human insulin analogue or derivative, polysaccharide, DNA, RNA, an enzyme, an oligonucleotide, an antiallergic, an antihistamine, an anti-inflammatory, a corticosteroid, a disease modifying antirheumatic drug, erythropoietin, and a vaccine.
